SEQUENCE LISTING (2 sequences)

SEQ ID NO: 1 - DNA, 1138 bp, synthetic construct
  source 1..1138: mol_type = other DNA
ttaactacgtaaaatcgccgctgctgtgtcctgatcggtaaccagtgcgttgatataaccgcctttcattgcagcggcaattgcttcggctttattttctccccctgccacgccaacccggacgggtatggtcttcagcgcgcttaaaggtaagccaatcagttcgttatgtattttgatattcgtgacaacgtcaccttttgcatcaaaaaagtagcctaaaatgtcgccaaccgccccttttcggccaatcattaactgttcgccctggctgatataaccggagcgaatgattgtcgcatcgtcctgttgactcacagcaccaatgccgacaatcgccacatccgctgcttgcgcggctaacagaacatctttgacgcaattttcattttttagcgtacgggcaatgtcagcggaggatgcccgcaacggagccggaataatattcacactgcacgccgcgttaagctgcccgattcccgtcatataagaaccgacgccaccggagagcgtgaccaggcgaatttgctgtgacgaaataaaaccacttaagcgttgcagcgtattcatggttgcctcgccaaaaccaatcgccagcatctgttgtggttgaagtaaactcatcaacatatgcgccgcgcctatccccagtcgcccaccgacatcagcatccgcaagcccagggatcacccggacatgttgcagcgaaaactgacgacgtaattgagtttcatattccagacagccttcaaagcgagaattaatctgtacgcgaataatgccggactgatgccctttctccagcaatcgcgacactttcaaacgtgtcaggccgagacgatcgctgatctcgctctgggtcagcccgtcgtgatagtaaaaccacgcgatccgcgcgacctgttcttcttcacacattccctgttctgaaattgccgaatcgttgattgtcatgaattcattaaagaggagaaaggtaccgctagcattatacctaggactgagctagctgtcaaaaatagcataaattgtgatctattcgtcggaaatatgtgcaatgtccacctaaggttatgaacaaattaaaagcagaaatacatttgttcaaaactcacctgcaaaactgaacgggggaaat

SEQ ID NO: 2 - DNA, 1202 bp, synthetic construct
  source 1..1202: mol_type = other DNA
ttaactacgtaaaatcgccgctgctgtgtcctgatcggtaaccagtgcgttgatataaccgcctttcattgcagcggcaattgcttcggctttattttctccccctgccacgccaacccggacgggtatggtcttcagcgcgcttaaaggtaagccaatcagttcgttatgtattttgatattcgtgacaacgtcaccttttgcatcaaaaaagtagcctaaaatgtcgccaaccgccccttttcggccaatcattaactgttcgccctggctgatataaccggagcgaatgattgtcgcatcgtcctgttgactcacagcaccaatgccgacaatcgccacatccgctgcttgcgcggctaacagaacatctttgacgcaattttcattttttagcgtacgggcaatgtcagcggaggatgcccgcaacggagccggaataatattcacactgcacgccgcgttaagctgcccgattcccgtcatataagaaccgacgccaccggagagcgtgaccaggcgaatttgctgtgacgaaataaaaccacttaagcgttgcagcgtattcatggttgcctcgccaaaaccaatcgccagcatctgttgtggttgaagtaaactcatcaacatatgcgccgcgcctatccccagtcgcccaccgacatcagcatccgcaagcccagggatcacccggacatgttgcagcgaaaactgacgacgtaattgagtttcatattccagacagccttcaaagcgagaattaatctgtacgcgaataatgccggactgatgccctttctccagcaatcgcgacactttcaaacgtgtcaggccgagacgatcgctgatctcgctctgggtcagcccgtcgtgatagtaaaaccacgcgatccgcgcgacctgttcttcttcacacattccctgttctgaaattgccgaatcgttgattgtcataattcattcttcactttgaacatatttaaatctttaatgcaattgttcagttcttgctcatttatatctgtgatggcaaccacagtttgactctacgagcatgaacaaacgcaaccgtgaaaatcaaaatagcataaattgtgatctattcgtcggaaatatgtgcaatgtccacctaaggttatgaacaaattaaaagcagaaatacatttgttcaaaactcacctgcaaaactgaacgggggaaat

(19) United States
(12) Patent Application Publication
(10) Pub. No.: US 2023/0167435 A1
(43) Pub. Date: Jun. 1, 2023

(54) AUTOINDUCER-2 (AI-2) MOLECULAR RESPONSE-BASED STARTING ELEMENT AND ESCHERICHIA COLI (E. COLI) DYNAMIC REGULATION SYSTEM AND METHOD CONSTRUCTED THEREBY

(71) Applicant: BRIGHT DAIRY & FOOD CO., LTD, Shanghai (CN)

(72) Inventors: Long LIU, Shanghai (CN); Jian CHEN, Shanghai (CN); Zhenmin LIU, Shanghai (CN); Xueqin LYU, Shanghai (CN); Guocheng DU, Shanghai (CN); Jianghua LI, Shanghai (CN); Miya SU, Shanghai (CN); Lu LIN, Shanghai (CN)

(73) Assignee: BRIGHT DAIRY & FOOD CO., LTD, Shanghai (CN)

(21) Appl. No.: 17/972,463

(22) Filed: Oct. 24, 2022

(30) Foreign Application Priority Data
Aug. 23, 2021 (CN) 202110968581.8

(51) Int. Cl. C12N 15/10; C12N 15/11; C12N 9/22; C12N 15/70; C12N 1/20

(52) U.S. Cl. CPC C12N 15/1086; C12N 15/11; C12N 9/22; C12N 15/70; C12N 1/20; C12N 2310/20; C12N 2800/80; C12N 2830/002; C12N 2800/101

(57) ABSTRACT

An autoinducer-2 (AI-2) molecular response-based starting element and an Escherichia coli (E. coli) dynamic regulation system and method constructed thereby are provided. A cell density-dependent starting element P_{J23119}-LsrR-P_{lsrA} based on an AI-2 molecular response is constructed. The element can be used to self-induce the expression of dCpf1, and crRNAs of different target genes are further assembled, such that the self-inducible element can be used for dCpf1-CRP to achieve the dynamic regulation of genes in a synthesis pathway. In the present disclosure, vectors pACYDuet-P_{J23119}-LsrR-P_{lsrA}-dCpf1-CRP, pRSFDuet-GFP-mCherry, and pETDuet-crRNA can be constructed to simultaneously achieve the transcriptional activation and inhibition of different genes. The construction method of recombinant E. coli in the present disclosure is simple and has promising application prospects.

### CROSS REFERENCE TO THE RELATED APPLICATIONS

This application is based upon and claims priority to Chinese Patent Application No. 202110968581.8, filed on Aug. 23, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBDZ045 Sequence Listing.xml, created on Oct. 24, 2022, and is 5,208 bytes in size.

### TECHNICAL FIELD

The present disclosure belongs to the technical field of genetic engineering and particularly relates to an autoinducer-2 (AI-2) molecular response-based starting element and an Escherichia coli (E. coli) dynamic regulation system and method constructed thereby.

### BACKGROUND

E. coli has long been widely used as a laboratory strain for molecular biology to facilitate the discovery of many important mechanisms in DNA replication, transcription, translation, and expression. In addition, E. coli is a preferred prokaryote for laboratory and industrial applications due to its rapid growth and development, simple cultivation conditions, strong metabolic plasticity, and rich biochemical and physiological functions. The relatively clear biochemical, physiological, and genetic properties of E. coli allow rapid progress in the research of metabolic engineering and synthetic biology, and E. coli has been widely used as an industrial host for the metabolic synthesis of a compound such as a lysine, 1,3-propanediol, and 1,4-butanediol. E. coli strains are generally considered to be harmless. For example, the laboratory strain E. coli MG1655 is considered to lack virulence factors associated with human diseases and has been widely used in the production of various biochemicals, food, and biofuels.

Programmable site-specific gene regulatory elements play an important role in genome-wide gene function research, metabolic pathway regulation, and artificial gene circuit design. Gene regulation tools based on clustered regularly interspaced short palindromic repeats (CRISPRs) are currently one of the most popular gene regulation techniques, and CRISPR technology can be used for the polygenic regulation of microorganisms. When a CRISPR gene regulation system guides the location of a defective nuclease (such as dCas9 and dCpf1) to a target gene site through a guide RNA (such as sgRNA and crRNA), DNA is not cleaved but is fused to a transcriptional repressor domain (TRD) to form an RNA-protein complex, and the RNA-protein complex is guided to a transcription start site of a target gene to inhibit the transcription. When a C terminus of the dCas protein is fused to a transcriptional activator, the dCas protein and the transcriptional activator can activate the transcription of the target gene at the transcription start site. In recent years, with the advent of programmable transcription factors for the CRISPR-dCpf1 system, the synthetic control of gene expression becomes simple. Compared with the CRISPR-dCas9 system, CRISPR-dCpf1 has significant advantages such as low off-target efficiency. In addition, the guide of dCpf1 only requires a single RNA, while the guide of dCas9 requires the joint action of sgRNA and tracrRNA. Therefore, the CRISPR-dCpf1 system is suitable for the regulation of multiple gene sites.

The dynamic regulation of gene expression has become a common regulatory strategy, and microorganisms can autonomously regulate their metabolic fluxes according to a microbial community or a cellular metabolism status. Quorum sensing (QS) is an information exchange mechanism by which bacteria monitor a population density and coordinate a microbial function by secreting signaling molecules, which has been widely used for automatic regulation of the expression of key genes in metabolic synthesis pathways or self-induction of the expression of recombinant proteins. An AI-2 signaling molecular response-based QS system (AI-2 QS) is an endogenous QS system in E. coli MG1655. In the AI-2 QS system, when a cell density is low, the transcription factor LsrR binds to an lsrA box sequence on a P_{lsrA} promoter, thereby inhibiting the transcription of the P_{lsrA} promoter. As bacterial cell density increases, a signaling molecule AI-2 synthesized in cells is continuously transported out of the cells through a TqsA protein. When the extracellular AI-2 signaling molecule accumulates to a specified threshold, the AI-2 molecule is transported into cells by the LsrABCD transport system and converted into a phosphorylated AI-2 molecule under the action of an LsrK protein, and the phosphorylated AI-2 molecule and the lsrA box sequence located on the P_{lsrA} promoter compete with each other to bind to the LsrR molecule, thereby releasing the transcriptional inhibition of LsrR on P and P_{lsrA} activating the expression of the promoter P_{lsrA} (FIG. 1). Therefore, a QS system of E. coli itself can be used to develop a cell density-dependent promoter P_{lsrA} to automatically initiate a CRISPR-dCpf1 regulation system, and then crRNAs of different genes in synthesis pathways can be further assembled for dynamic regulation of genes in the metabolic synthesis pathways.

The CRISPR-dCpf1 system is currently a popular gene editing and regulation system, which requires a shorter guide RNA (crRNA) sequence than the CRISPR-dCas9 system and can achieve the polygenic regulation by constructing an array of different crRNAs. Miao Chengsi et al. investigated the influence of a target sequence position, lengths of repeat and spacer sequences in crRNA, a protospacer adjacent motif (PAM) sequence, and the like in the CRISPR-dCpf1 system on the regulation performance of the CRISPR-dCpf1 system in E. coli and discovered an efficient CRISPR-dCpf1 gene regulation tool based on isopropyl-β-D-thiogalactoside (IPTG) induction. Wu Yaokang et al. also constructed a xylose induction-based CRISPR/Cpf1 polygenic editing and transcriptional regulation system (CAMERS-B) in Bacillus subtilis (B. subtilis) by designing a crRNA array, which can not only achieve the double-gene knockout, multi-point mutation, or single-gene insertion in B. subtilis but also achieve the transcriptional inhibition and activation of multiple genes. The CAMERS-B system can be further used to inhibit the genes (bdhA and acoA) of an acetoin catabolism pathway and the genes (ldh and pta) of the synthesized byproducts of lactic acid and acetic acid and to activate the pathway gene alsSD in the acetoin synthesis pathway and the gene alsR of a transcriptional activator. A crRNA array for bdhA, acoA, ldh, pta, and alsR is constructed to achieve the transcriptional inhibition and activation of the CAMERS-B system in acetoin synthesis-associated pathways, thereby increasing a yield of the acetoin by 44.8% and to 25.8 g/L.

Although a CRISPR-dCpf1 regulation system based on IPTG induction has been developed, the induction of overexpression of dCpf1 by IPTG may cause a metabolic burden on cells, thereby negatively affecting metabolic synthesis. Therefore, if a self-inducible signaling molecule is used to automatically trigger the CRISPR-dCpf1 regulation system, the metabolic burden of the recombinant protein on cells and the potential toxic effect of IPTG on cells can be avoided. When some exogenous metabolic synthesis pathways are introduced, it is difficult to develop a self-inducible element that can respond to a specific signaling molecule for dynamic regulation of the expression of key genes in a synthesis pathway due to a specified threshold concentration range for responding to an intermediate metabolite or due to the lack of research on regulation mechanisms for responding to an intermediate metabolite. The dynamic regulation based on cell QS is currently a regulation system with the potential for metabolic synthesis. Without responding to a specific intermediate metabolite, the system can secrete a relevant signaling molecule through a change in a cell population density to activate or inhibit the expression of a related gene. Therefore, an endogenous cell density-dependent AI-2 signaling molecule of E. coli can be used to develop a self-inducible dCpf1 expression element, and crRNA of a specific gene can be further constructed to achieve the dynamic activation or inhibition of the expression of related genes in the metabolic synthesis pathway.

### SUMMARY

To solve the problem that the IPTG induction-based CRISPR-dCpf1 regulation system may have a potential toxic effect on cells, the present disclosure constructs a cell density-dependent response element P_{J23119}-LsrR-P_{lsrA} (SEQ ID NO: 1) based on an AI-2 signaling molecule. The element can self-induce the expression of dCpf1, and crRNA of a specific gene can be further constructed to dynamically activate or inhibit the expression of related genes in a metabolic synthesis pathway, thereby avoiding the potential toxic effect of an inducer such as IPTG on cells. The present disclosure mainly adopts the following technical solutions to solve the above technical problem:

The present disclosure provides an AI-2 molecular response-based starting element, where the AI-2 molecular response-based starting element is a cell density-dependent starting element P_{J23119}-LsrR-P_{lsrA}. The cell density-dependent starting element P_{J23119}-LsrR-P_{lsrA} is constructed as follows: optimizing a native cell density-dependent promoter P_{lsrA} in an AI-2 signaling molecular response-based wild-type (WT) starting element gene fragment P_{lsrR}-LsrR-P_{lsrA}, using a constitutive promoter P_{J23119} to express a transcriptional regulation factor LsrR, and constructing the cell density-dependent starting element P_{J23119}-LsrR-P_{lsrA} based on an AI-2 molecular response. Preferably, the starting element P_{J23119}-LsrR-P_{lsrA} has a nucleotide sequence shown in SEQ ID NO: 1, and the WT starting element gene fragment P_{lsrR}-LsrR-P_{lsrA} has a nucleotide sequence shown in SEQ ID NO: 2.

The present disclosure also provides a use of a cell density-dependent starting element P_{J23119}-LsrR-P_{lsrA} based on an AI-2 molecular response for automatically triggering a CRISPR-dCpf1 regulation system.

The present disclosure also provides an AI-2 molecular response-based E. coli CRISPR-dCpf1 dynamic regulation system, including a dCpf1 protein expression vector pACYDuet-dCpf1, a reporter fluorescent protein GFP, an mCherry expression vector pRSFDuet-GFP-mCherry, and a crRNA expression vector pETDuet-crRNA of a reporter protein. Preferably, the expression vector pACYDuet-dCpf1 includes a cell density-dependent starting element P_{J23119}-LsrR-P_{lsrA}.

Preferably, a transcriptional activator CRP in E. coli is selected and fused to a C terminus of a dCpf1 protein.

The present disclosure also provides a construction method of an AI-2 molecular response-based E. coli CRISPR-dCpf1 dynamic regulation system, including the following steps:

(1) selecting plasmids pACYDuet, pRSFDuet, and pETDuet as starting vectors, recombinantly ligating FndCpf1 and P_{J23119}-LsrR-P_{lsrA} gene fragments with pACYDuet to construct a vector pACYDuet-P_{J23119}-LsrR-P_{lsrA}-dCpf1, recombinantly ligating a GFP gene with a linearized vector pRSFDuet to construct a vector pRSFDuet-GFP, and recombinantly ligating a crRNA fragment with a linearized vector pETDuet to construct a crRNA gene-containing vector pETDuet-crRNA;

(2) with pACYDuet-P_{J23119}-LsrR-P_{lsrA}-dCpf1 as a template, fusing a transcriptional activator CRP to a C terminus of a dCpf1 protein to construct a dCpf1 activation plasmid pACYDuet-P_{J23119}-LsrR-P_{lsrA}-dCpf1-CRP;

(3) with the vector pRSFDuet-GFP obtained in step (1) as a template, recombinantly ligating a gene fragment of mCherry with pRSFDuet-GFP to construct a reporter vector pRSFDuet-GFP-mCherry for fluorescence activation and inhibition; and

(4) co-transforming the plasmid pACYDuet-P_{J23119}-LsrR-P_{lsrA}-dCpf1-CRP into an E. coli MG1655 competent cell with the vectors pRSFDuet-GFP-mCherry and pETDuet-crRNA.

The present disclosure also provides a use of an AI-2 molecular response-based E. coli CRISPR-dCpf1 dynamic regulation system in the regulation of expression of a synthesis pathway gene in E. coli.

Compared with the prior art, the present disclosure has the following positive progressive effects:

The present disclosure constructs a cell density-dependent starting element P_{J23119}-LsrR-P_{lsrA} based on an AI-2 molecular response. A strong promoter P_{J23119} is used to express a transcription factor LsrR, which can effectively enhance a fluorescence intensity of a reporter protein. By assembling crRNAs of different target genes, the self-inducible element can be used for dCpf1-CRP to achieve the dynamic regulation of genes in a synthesis pathway. In the present disclosure, vectors pACYDuet-P_{J23119}-LsrR-P_{lsrA}-dCpf1-CRP, pRSFDuet-GFP-mCherry, and pETDuet-crRNA can be constructed to simultaneously achieve the transcriptional activation and inhibition of different genes. The construction method of recombinant E. coli in the present disclosure is simple and has promising application prospects.

### BRIEF DESCRIPTION OF THE DRAWINGS

FIG. 1 shows an AI-2 signaling molecular response-based E. coli QS system;

FIG. 2 is a schematic diagram illustrating the construction of a CRISPR-dCpf1 regulation system;

FIG. 3 shows the activation intensities of transcription factors CRP and RpoZ on a green fluorescent protein (GFP);

FIG. 4 shows the regulatory effects of arabinose induction-based CRISPR-dCpf1 on the activation and inhibition of genes;

FIG. 5 shows the fluorescence intensity of a cell density-dependent promoter P_{lsrA} based on an AI-2 signaling molecule; and

FIG. 6 shows the regulatory effects of cell density-dependent CRISPR-dCpf1 on the activation and inhibition of genes.

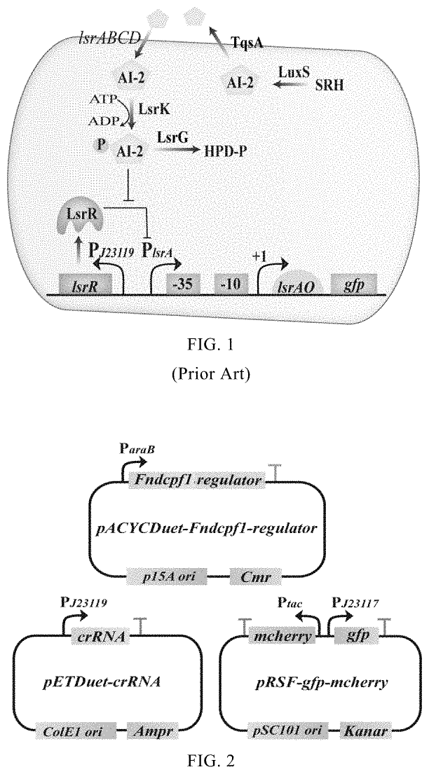

### DETAILED DESCRIPTION OF THE EMBODIMENTS

The present disclosure provides a cell density-dependent element P_{J23119}-LsrR-P_{lsrA} based on an AI-2 signaling molecular response, and the element can be used to automatically trigger a CRISPR-dCpf1 regulation system, which provides a tool for dynamic regulation of a gene in a metabolic synthesis pathway. In the present disclosure, the element P_{J23119}-LsrR-P_{lsrA} has a nucleotide sequence shown in SEQ ID NO: 1.

In the present disclosure, to construct a CRISPR-dCpf1 system with a regulatory effect for activation and inhibition, 3 compatible plasmids are first constructed, including a dCpf1 protein expression vector pACYDuet-dCpf1, a reporter fluorescent protein GFP, an mCherry expression vector pRSFDuet-GFP-mCherry, and a crRNA expression vector pETDuet-crRNA of a reporter protein. Transcriptional activators (CRP and RpoZ) in E. coli are selected and then each fused to a C terminus of a dCpf1 protein, and the activation intensities of different transcription factors are detected.

In the present disclosure, since a native cell density-dependent promoter P_{lsrA} based on an AI-2 signaling molecular response has a low fluorescence intensity, the promoter needs to be optimized, including replacement of a core region of the promoter, random mutation of a ribosome binding site (RBS) sequence, and expression of a transcription factor LsrR. Subsequently, the arabinose induction-based promoter P_{araB} in the CRISPR-dCpf1 system is replaced with the cell density-dependent promoter P_{lsrA} to construct a CRISPR-dCpf1 dynamic regulation system with both activation and inhibition effects. With the AI-2 molecular response-based CRISPR-dCpf1 dynamic regulation system constructed in the present disclosure, the expression of synthesis pathway genes in E. coli can be dynamically regulated by simply designing and assembling crRNAs of metabolic synthesis pathway-associated genes.

The present disclosure also provides a construction method of an AI-2 signaling molecule response-based E. coli CRISPR-dCpf1 dynamic regulation system, including the following steps:

### (1) Construction of a CRISPR-dCpf1 Activation System

The construction of the CRISPR-dCpf1 regulation system is shown in FIG. 2. The plasmids pACYDuet, pRSFDuet, and pETDuet commonly used for E. coli are selected as starting vectors. The gene fragments of FndCpf1 and araB inducing elements (araC-P_{araB}) are recombinantly ligated with pACYDuet using a seamless cloning kit to construct a vector pACYDuet-P_{araB}-dCpf1 with arabinose to induce the expression of dCpf1. The GFP gene is recombinantly ligated with a linearized vector pRSFDuet, and a high-copy replicon pRSF ori on the initial vector pRSFDuet is replaced with a low-copy replicon SC101 ori to construct a vector pRSFDuet-GFP. A crRNA fragment is recombinantly ligated with a linearized vector pETDuet to construct a vector pETDuet-crRNA with P_{J23119} to express crRNA. With pACYDuet-P_{araB}-dCpf1 as a template, the transcriptional activators CRP and RpoZ are each fused to a C terminus of a dCpf1 protein to construct two dCpf1 activation plasmids pACYDuet-P_{araB}-dCpf1-CRP and pACYDuet-P_{araB}-dCpf1-RpoZ. The dCpf1 expression plasmids pACYDuet-P_{araB}-dCpf1, pACYDuet-Parma-dCpf1-CRP, and pACYDuet-P_{araB}-dCpf1-RpoZ are each co-transformed into an E. coli MG1655 competent cell with the vectors pRSFDuet-GFP and pETDuet-crRNA, and resulting single colonies are picked and cultivated in a 96-well plate to investigate the activation effects of these two transcription factors.

### (2) Construction of a CRISPR-dCpf1-CRP Activation and Inhibition System

With the vector pRSFDuet-GFP obtained in step (1) as a template, a gene fragment of mCherry is recombinantly ligated with pRSFDuet-GFP using a seamless cloning kit to construct a reporter vector pRSFDuet-GFP-mCherry for fluorescence activation and inhibition. The transcription factor CRP vector pACYDuet-P_{araB}-dCpf1-CRP with high fluorescence intensity in step (1) is co-transformed into an E. coli MG1655 competent cell with the vectors pRSFDuet-GFP-mCherry and pETDuet-crRNA, and resulting single colonies are picked and cultivated in a 96-well plate to verify that the CRISPR-P_{araB}-dCpf1-CRP system can achieve both activation and inhibition effects.

### (3) Construction of a Cell Density-Dependent Element Based on an AI-2 Signaling Molecular Response

The P_{araB} promoter gene fragment and P_{lsrA} ID promoter gene fragment (SEQ NO: 2) on the E. coli MG1655 genome are each recombinantly ligated with the vector pACYDuet using a seamless cloning kit to construct control vectors pACYDuet-P_{araB}-gfp and pACYDuet-P_{lsrR}-LsrR-P_{lsrA}-gfp, respectively. Since a native cell density-dependent promoter P_{lsrA} based on an AI-2 signaling molecular response has a low fluorescence intensity, the transcription factor LsrR is further expressed using a constitutive promoter P_{J23119} to construct a vector pACYDuet-P_{J23119}-LsrR-P_{lsrA}-gfp. The pACYDuet-P_{araB}-gfp pACYDuet-P_{lsrR}-LsrR-P_{lsrA}-gfp, and pACYDuet-P_{J23119}-LsrR-P_{lsrA}-gfp plasmids are each transformed into an E. coli MG1655 competent cell, and resulting single colonies are picked and cultivated in a 96-well plate to compare the fluorescence intensities of the cell density-dependent promoter P_{lsrA} and the inducible promoter P_{araB}.

### (4) Construction of a CRISPR-dCpf1-CRP Dynamic Regulation System

The arabinose-induced promoter P_{araB} in the CRISPR-dCpf1 system is replaced with each of the gene sequences of P_{lsrR}-LsrR-P_{lsrA} and P_{J23119}-LSIR-P_{lsrA} to construct plasmids pACYDuet-P_{lsrR}-LsrR-P_{lsrA}-dCpf1 -CRP and pACYDuet-P_{J23119}-LsrR-P_{lsrA}-dCpf1 -CRP, respectively. The two plasmids are each co-transformed into an E. coli MG1655 competent cell with the vectors pRSFDuet-GFP-mCherry and pETDuet-crRNA, and resulting single colonies are picked and cultivated in a 96-well plate to verify that CRISPR-P_{lsrA}-dCpf1-CRP can spontaneously initiate the effects of activating and inhibiting a gene according to a change in cell density.

To make the objectives, technical solutions, and advantages of the present disclosure clear, the present disclosure will be described in detail below in conjunction with examples, but the examples should not be construed as limiting the protection scope of the present disclosure.

### Example 1

### (1) Construction of a CRISPR-dCpf1 Activation System

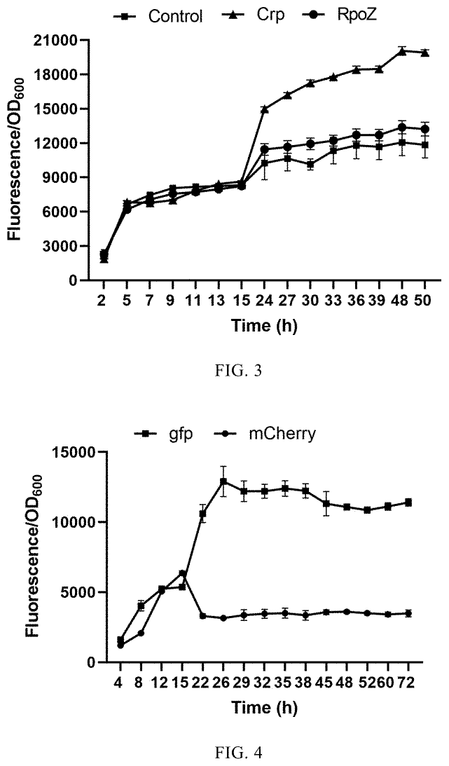

The plasmids pACYDuet, pRSFDuet, and pETDuet commonly used by E. coli were selected as starting vectors, each was linearized by polymerase chain reaction (PCR), and the template DNA was digested with a DpnI enzyme. An FndCpf1 gene was amplified with the pLCg6-dCpf1 plasmid in Chinese Patent CN201911387447.8 as a template. The arabinose promoter P_{araB} was amplified with the E. coli genome as a template; purified and recovered fragments of FndCpf1 and P_{araB} were ligated to pACYDuet using a seamless cloning kit to construct a vector pACYDuet-P_{araB}-dCpf1. The gfp gene in Chinese Patent CN201911387447.8 was amplified. A promoter P_{J23117} and an upstream sequence thereof (from the literature: Bikard, D.; Jiang, W.; Samai, P.; Hochschild, A.; Zhang, F.; Marraffini, L. A., Programmable repression and activation of bacterial gene expression using an engineered CRISPR-Cas system. Nucleic Acids Res. 2013, 41 (15), 7429-7437.) were acquired through gene synthesis, and the two fragments were recombinantly ligated with a linearized vector pRSFDuet. The replicon pSC101 ori was amplified with a plasmid pSC101-Donor (Addgene #140630) as a template to construct a low-copy reporter protein plasmid pRSFDuet-GFP. A crRNA fragment was amplified with the perF11 vector in Chinese Patent CN201911387447.8 as a template and then recombinantly ligated with pETDuet through seamless cloning, then crRNA of an upstream sequence of the gfp gene promoter was designed on the CRISPR-DT web site (http://bioinfolab.miamioh.edu/CRISPR-DT/interface/Cpf1_design.php), and a vector pETDuet-crRNA for activating the gfp gene was constructed. The vector pACYDuet-P_{araB}-dCpf1 was then linearized, gene fragments of the transcriptional activators CRP and RpoZ were each amplified with the E. coli genome as a template, and the transcription factors were each fused to a C terminus of a dCpf1 protein through a (GGGGS)₂ flexible Linker to construct dCpf1 activation plasmids pACYDuet-P_{araB}-dCpf1-CRP and pACYDuet-P_{araB}-dCpf1-RpoZ, respectively. The dCpf1 expression plasmids pACYDuet-P_{araB}-dCpf1, pACYDuet-Parma-dCpf1-CRP, and pACYDuet-P_{araB}-dCpf1-RpoZ were each co-transformed into an E. coli MG1655 competent cell with the vectors pRSFDuet-GFP and pETDuet-crRNA, then a resulting competent cell was coated on a chloramphenicol, kanamycin, and ampicillinum-resistant plate and cultivated at 30°C. for 16 h to 18 h. Positive single colonies were picked and cultivated in a 96-well plate, and activation intensities of the two transcription factors (CRP and RpoZ) on a GFP were detected by a Tecan microplate reader. An excitation wavelength of the GFP was set as 490 mm, and an emission wavelength was set as 530 mm. As shown in FIG. 3, an activation effect of the transcription factor RpoZ on GFP was not significant, while an activation effect of the transcription factor CRP on GFP was significant. The green fluorescence intensity was increased by 1.8 times compared with the case without the fusion of a transcription factor.

### (2) Construction of a CRISPR-dCpf1-CRP Activation and Inhibition System

The plasmid pRSFDuet-GFP was linearized by PCR, the template DNA was digested with a DpnI enzyme, the red fluorescent protein (RFP) gene mCherry in Chinese Patent CN201911387447.8 was amplified, and the gene fragment of mCherry was recombinantly ligated with a linearized vector pRSFDuet-GFP using a seamless cloning kit to construct a reporter vector pRSFDuet-GFP-mCherry for fluorescence activation and inhibition. The vector pACYDuet-P_{araB}-dCpf1-CRP with high activation intensity for the transcription factor CRP (FIG. 3) was selected and co-transformed into an E. coli MG1655 competent cell with the reporter protein plasmids pRSFDuet-GFP-mCherry and pETDuet-crRNA. The competent cell was coated on a chloramphenicol, kanamycin, and ampicillinum-resistant plate and cultivated at 30°C. for 16 h to 18 h. Positive single colonies were picked and cultivated in a 96-well plate for 12 h to 15 h, and arabinose was added at a final concentration of 40 mM to 60 mM to induce the expression of GFP and mCherry. The expression intensities of GFP and RFP were detected using a Tecan microplate reader. An excitation wavelength of GFP was set as 490 mm and an emission wavelength of GFP was set as 530 mm; an excitation wavelength of RFP was set as 580 mm and an emission wavelength of RFP was set as 615 mm. As shown in FIG. 4, after arabinose was added to induce the expression of dCpf1, starting from 15 h, the expression intensity of GFP was significantly increased, while the expression intensity of RFP was gradually decreased. It indicates that the constructed CRISPR-dCpf1 regulation system can simultaneously activate and inhibit the expression intensities of different genes under the action of arabinose induction.

### Example 2

### Construction of a Vector pACYDuet-P_{lsrR}-LsrR-P_{lsrA}-gfP

The plasmid pACYDuet was selected as a starting plasmid and linearized by PCR, and the template DNA was digested with a DpnI enzyme. A WT arabinose promoter P_{araB} and a cell density-dependent promoter P_{lsrA} were each amplified with an E. coli genome as a template, and purified and recovered fragments of P_{araB} and P_{lsrA} were ligated to a linearized vector pACYDuet using a seamless cloning kit to construct GFP reporter vectors pACYDuet-P_{araB}-gfp and pACYDuet-P_{lsrR}-LsrR-P_{lsrA}-gfp, respectively. The constructed plasmids pACYDuet-P_{araB}-gfp and pACYDuet-P_{lsrR}-LsrR-P_{lsrA}-gfp were each transformed into an E. coli MG1655 competent cell, and resulting single colonies were picked and cultivated in a 96-well plate to compare the fluorescence intensities of the cell density-dependent promoter P_{lsrA} and the inducible promoter P_{araB}. The expression intensities of GFP were detected using a Tecan microplate reader. An excitation wavelength of GFP was set as 490 mm, and an emission wavelength of GFP was set as 530 mm.

### Example 3

### Construction of a Vector pACYDuet-P_{J23119}-LsrR-P_{lsrA}-gfp

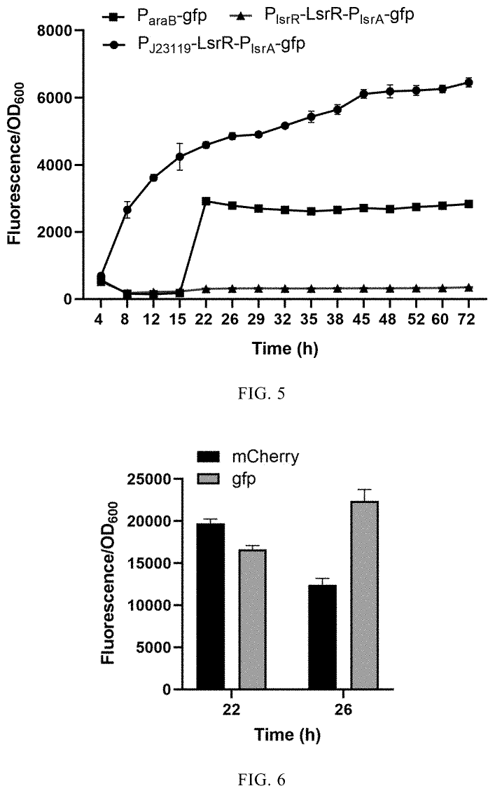

Since the pACYDuet-P_{lsrR}-LsrR-P_{lsrA}-gfp had a weak fluorescence intensity when the transcription factor LsrR was expressed using a WT promoter P_{lsrR}, a constitutive promoter P_{J23119} was selected to express the transcriptional regulation factor LsrR. With the plasmid pACYDuet-P_{lsrR}-LsrR-P_{lsrA}-gfp as a template, the WT P_{lsrR} promoter was replaced with a P_{J23119} promoter through cyclic PCR, and the template DNA was digested with a DpnI enzyme. Purified and recovered fragments were transformed into an E. coli MG1655 competent cell. The competent cell was coated on a chloramphenicol-resistant plate and cultivated overnight at 37°C., and positive single colonies were picked for testing. The plasmid pACYDuet-P_{J23119}-LsrR-P_{lsrA}-gfp sequenced as positive and the plasmids pACYDuet-P_{araB}-gfp and pACYDuet-P_{lsrR}-LsrR-P_{lsrA}-gfp successfully constructed in Example 2 were each transformed into an E. coli MG1655 competent cell once again. The competent cell was coated on a chloramphenicol-resistant plate, and single colonies were picked and cultivated in a 96-well plate. An excitation wavelength of GFP was set as 490 mm, and an emission wavelength of GFP was set as 530 mm. These three plasmids were compared in terms of the expression intensity of GFP. As shown in FIG. 5, the WT P_{lsrA} promoter had the weakest intensity and was barely expressed. After the transcription factor LsrR was expressed using the constitutive promoter P_{J23119}, a GFP fluorescence intensity was 2.2 times higher than a GFP fluorescence intensity of the inducible promoter P_{araB}.

### Example 4

### Construction of a CRISPR-dCpf1-CRP Dynamic Regulation System

The plasmid pACYDuet-P_{araB}-dCpf1-CRP constructed in Example 1 was linearized by PCR, and the template DNA was digested with a DpnI enzyme. A gene fragment P_{J23119}-LsrR-P_{lsrA} was amplified with the plasmid pACYDuet-P_{J23119}-LsrR-P_{lsrA}-gfp constructed in Example 3 as a template. Purified and recovered fragments of P_{J23119}-LsrR-P_{lsrA} were ligated to a linearized vector pACYDuet-P_{araB}-dCpf1-CRP using a seamless cloning kit, and the arabinose-induced promoter P_{araB} in the CRISPR-dCpf1 system was replaced with the cell density-dependent starting element P_{J23119}-LsrR-P_{lsrA} constructed in Example 3 to construct a plasmid pACYDuet-P_{J23119}-LsrR-P_{lsrA}-dCpf1-CRP. The plasmid pACYDuet-P_{J23119}-LsrR-P_{lsrA}-dCpf1-CRP was co-transformed into an E. coli MG1655 competent cell with the reporter protein plasmids pRSFDuet-GFP-mCherry and pETDuet-crRNA. The competent cell was coated on a chloramphenicol-resistant plate, and single colonies were picked and cultivated in a 96-well plate. An excitation wavelength of GFP was set as 490 mm, and an emission wavelength of GFP was set as 530 mm; an excitation wavelength of RFP was set as 580 mm, and an emission wavelength of RFP was set as 615 mm. It was verified that CRISPR-P_{lsrA}-dCpf1-CRP could spontaneously activate GFP and inhibit RFP according to a change in cell density. As shown in FIG. 6, after the cell was cultivated for 22 h to 26 h, the CRISPR-dCpf1 dynamic regulation system exhibited significant activation (1.3 times) and inhibition (1.5 times) effects on GFP and RFP.

The above are merely preferred examples of the present disclosure and are not intended to limit the present disclosure, and various changes and modifications can be made to the present disclosure by those skilled in the art. Any modifications, equivalent substitutions, improvements, and the like made within the spirit and principle of the present disclosure should be included within the protection scope of the present disclosure.

What is claimed is:

1. An autoinducer-2 (AI-2) molecular response-based starting element, wherein the AI-2 molecular response-based starting element is a cell density-dependent starting element P_{J23119}-LsrR-P_{lsrA}; the cell density-dependent starting element P_{J23119}-LsrR-P_{lsrA} is constructed as follows: optimizing a native cell density-dependent promoter P_{lsrA} in an AI-2 signaling molecular response-based wild-type (WT) starting element gene fragment P_{lsrR}-LsrR-P_{lsrA}, using a constitutive promoter P_{J23119} to express a transcriptional regulation factor LsrR, and constructing the cell density-dependent starting element P_{J23119}-LsrR-P_{lsrA} based on an AI-2 molecular response; and the cell density-dependent starting element P_{J23119}-LsrR-P_{lsrA} has a nucleotide sequence shown in SEQ ID NO: 1, and the AI-2 signaling molecular response-based WT starting element gene fragment P_{lsrR}-LsrR-P_{lsrA} has a nucleotide sequence shown in SEQ ID NO: 2.

2. A method of a use of a cell density-dependent starting element P_{J23119}-LsrR-P_{lsrA} based on an AI-2 molecular response for automatically triggering a CRISPR-dCpf1 regulation system.

3. An AI-2 molecular response-based Escherichia coli (E. coli) CRISPR-dCpf1 dynamic regulation system, comprising a dCpf1 protein expression vector pACYDuet-dCpf1, a reporter fluorescent protein GFP, an mCherry expression vector pRSFDuet-GFP-mCherry, and a crRNA expression vector pETDuet-crRNA of a reporter protein, wherein the dCpf1 protein expression vector pACYDuet-dCpf1 comprises a cell density-dependent starting element P_{J23119}-LsrR-P_{lsrA}.

4. The AI-2 molecular response-based E. coli CRISPR-dCpf1 dynamic regulation system according to claim 3, wherein a transcriptional activator CRP in E. coli is selected and fused to a C terminus of a dCpf1 protein.

5. A construction method of an AI-2 molecular response-based E. coli CRISPR-dCpf1 dynamic regulation system, comprising the following steps:
(1) selecting plasmids pACYDuet, pRSFDuet, and pETDuet as starting vectors; recombinantly ligating FndCpf1 and P_{J23119}-LsrR-P_{lsrA} gene fragments with the plasmid pACYDuet to construct a vector pACYDuet-P_{J23119}-LsrR-P_{lsrA}-dCpf1, recombinantly ligating a GFP gene with a linearized vector pRSFDuet to construct a vector pRSFDuet-GFP, and recombinantly ligating a crRNA fragment with a linearized vector pETDuet to construct a crRNA gene-containing vector pETDuet-crRNA;
(2) with the vector pACYDuet-P_{J23119}-LsrR-P_{lsrA}-dCpf1 as a template, fusing a transcriptional activator CRP to a C terminus of a dCpf1 protein to construct a dCpf1 activation plasmid pACYDuet-P_{J23119}-LsrR-P_{lsrA}-dCpf1 -CRP;
(3) with the vector pRSFDuet-GFP obtained in step (1) as a template, recombinantly ligating a gene fragment of mCherry with the vector pRSFDuet-GFP to construct a reporter vector pRSFDuet-GFP-mCherry for fluorescence activation and inhibition; and
(4) co-transforming the dCpf1 activation plasmid pACYDuet-P_{J23119}-LsrR-P_{lsrA}-dCpf1 -CRP into an E. coli MG1655 competent cell with the reporter vector pRSFDuet-GFP-mCherry and the crRNA gene-containing vector pETDuet-crRNA.

6. A method of a use of an AI-2 molecular response-based E. coli CRISPR-dCpf1 dynamic regulation system in a regulation of an expression of a synthesis pathway gene in E. coli.
